(19) United States
(12) Patent Application Publication
(10) Pub. No.: US 2023/0154608 A1
(43) Pub. Date: May 18, 2023

(54) MACHINE LEARNING TECHNIQUES FOR PREDICTIVE ENDOMETRIOSIS-BASED PREDICTION

(71) Applicant: Optum, Inc., Minnetonka, MN (US)

(72) Inventor: Priyanka Singh Gunsola, Greater Noida (IN)

(21) Appl. No.: 17/455,303

(22) Filed: Nov. 17, 2021

(51) Int. Cl. G16H 50/20; G16H 10/60; G16H 50/30

(52) U.S. Cl. CPC G16H 50/20; G16H 10/60; G16H 50/30

(57) ABSTRACT

Various embodiments of the present invention utilize systems, methods, and computer program products that perform predictive data analysis operations by determining, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; and in response to determining that the inferred endometriosis prediction for the selected diagnostic profile classification is an affirmative inferred endometriosis prediction: determining, using a recommendation classification machine learning model, and based at least in part on a plurality of recommendation feature values, a selected recommendation profile classification of a plurality of defined recommendation profile classifications.

### BACKGROUND

Various embodiments of the present invention address technical challenges related to performing predictive data analysis operations and address the efficiency and reliability shortcomings of various existing predictive data analysis solutions, in accordance with at least some of the techniques described herein.

### BRIEF SUMMARY

In general, embodiments of the present invention provide methods, apparatus, systems, computing devices, computing entities, and/or the like for performing predictive data analysis operations. For example, certain embodiments of the present invention utilize systems, methods, and computer program products that perform predictive data analysis operations by determining, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; and in response to determining that the inferred endometriosis prediction for the selected diagnostic profile classification is an affirmative inferred endometriosis prediction: determining, using a recommendation classification machine learning model, and based at least in part on a plurality of recommendation feature values, a selected recommendation profile classification of a plurality of defined recommendation profile classifications.

In accordance with one aspect, a method is provided. In one embodiment, the method comprises: determining, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; in response to determining that the inferred endometriosis prediction for the selected diagnostic profile classification is an affirmative inferred endometriosis prediction: (i) determining a plurality of recommendation feature values for the monitored individual based at least in part on the plurality of diagnostic feature values for the monitored individual and the inferred endometriosis diagnosis history prediction for the selected diagnostic profile classification, (ii) determining, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action, (iii) determining a predicted endometriosis-based recommendation based at least in part on the recommended endometriosis-based action for the selected recommendation profile classification, and (iv) performing one or more recommendation-based actions based at least in part on the predicted endometriosis-based recommendation.

In accordance with another aspect, a computer program product is provided. The computer program product may comprise at least one computer-readable storage medium having computer-readable program code portions stored therein, the computer-readable program code portions comprising executable portions configured to: determine, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; in response to determining that the inferred endometriosis prediction for the selected diagnostic profile classification is an affirmative inferred endometriosis prediction: (i) determine a plurality of recommendation feature values for the monitored individual based at least in part on the plurality of diagnostic feature values for the monitored individual and the inferred endometriosis diagnosis history prediction for the selected diagnostic profile classification, (ii) determine, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action, (iii) determine a predicted endometriosis-based recommendation based at least in part on the recommended endometriosis-based action for the selected recommendation profile classification, and (iv) perform one or more recommendation-based actions based at least in part on the predicted endometriosis-based recommendation.

In accordance with yet another aspect, an apparatus comprising at least one processor and at least one memory including computer program code is provided. In one embodiment, the at least one memory and the computer program code may be configured to, with the processor, cause the apparatus to: determine, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; in response to determining that the inferred endometriosis prediction for the selected diagnostic profile classification is an affirmative inferred endometriosis prediction: (i) determine a plurality of recommendation feature values for the monitored individual based at least in part on the plurality of diagnostic feature values for the monitored individual and the inferred endometriosis diagnosis history prediction for the selected diagnostic profile classification, (ii) determine, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action, (iii) determine a predicted endometriosis-based recommendation based at least in part on the recommended endometriosis-based action for the selected recommendation profile classification, and (iv) perform one or more recommendation-based actions based at least in part on the predicted endometriosis-based recommendation.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example client computing entity in accordance with some embodiments discussed herein.

FIG. 4 is a flowchart diagram of an example process for generating a predicted endometriosis-based recommendation for a monitored individual in accordance with one or more optimal imbalance adjustment conditions in accordance with some embodiments discussed herein.

FIG. 5 is a flowchart diagram of an example process for generating a predicted endometriosis-based recommendation for a monitored individual based at least in part on the selected diagnostic profile classification for the monitored individual in accordance with some embodiments discussed herein.

FIG. 6 provides an operational example of a recommendation output user interface in accordance with some embodiments discussed herein.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis tasks.

### I. Overview and Technical Improvements

Various embodiments of the present invention introduce techniques for training diagnosis classification machine learning models and/or recommendation classification machine learning models without using target training data describing diagnosis labels and/or recommendation labels. By doing so, various embodiments of the present invention reduce the storage-wise complexity and thus improve storage-wise efficiency of predictive data analysis systems that are configured to train and use diagnosis classification machine learning models and/or recommendation classification machine learning models by reducing the amount of training data those systems need to store to train diagnosis classification machine learning models and/or recommendation classification machine learning models. Moreover, by enabling techniques for training diagnosis classification machine learning models and/or recommendation classification machine learning models without using target training data describing diagnosis labels and/or recommendation labels, various embodiments of the present invention enable accurate and efficient machine learning even in the absence of target training data describing diagnosis labels and/or recommendation labels, thus expanding the utility of the resulting models and the environments in which those models can be utilized. Accordingly, various embodiments of the present invention improve efficiency and reliability of predictive data analysis systems that are configured to train and use diagnosis classification machine learning models and/or recommendation classification machine learning models, and make important technical contributions to the field of predictive data analysis.

For example, in some embodiments, the diagnostic classification machine learning model may be configured to process a plurality of diagnostic feature values (e.g., an n-dimensional vector comprising n diagnostic feature values) for a monitored individual to generate a model output value that can be used to select a defined diagnostic profile classification for the monitored individual. In some embodiments, the diagnostic classification machine learning model is a powerful supervised machine learning tool that can be trained without labeled training data describing desired target information yet can be configured to generate predictive outputs that can be used to probabilistically infer the desired target information. For example, the diagnostic classification machine learning model can generate predictions about whether a monitored individual is associated with an endometriosis diagnosis history (i.e., whether the monitored has in the past been diagnosed with endometriosis) and/or whether a monitored individual is suffering from endometriosis without being trained on labeled training data describing endometriosis diagnosis history labels and/or endometriosis diagnosis labels. To do so, the diagnostic classification machine learning model can be trained to map monitored individuals to defined diagnostic profile classifications instead of to target diagnosis history predictions and/or to target diagnosis predictions, and then the diagnostic profile classifications may be used to infer distributions for target diagnosis history predictions and/or to target diagnosis predictions that can be used to generating a predicted endometriosis-based recommendation for a monitored individual.

To illustrate an exemplary application of the above-described point, consider a predictive task that seeks to recommend endometriosis-based actions (e.g., endometriosis-based medical interventions) for those monitored individuals that are likely suffering from endometriosis without labeled data describing whether particular monitored individuals are suffering from endometriosis and/or without labeled data describing what recommended endometriosis-based actions are for particular monitored individuals that are suffering from endometriosis. To do so, the diagnostic classification machine learning model may be trained to map a monitored individual to a selected diagnostic profile classification which can be based at least in part on medical history data for the monitored individual (and thus may be readily determinable during training data preparation process), where the selected diagnostic profile classification for the monitored individual is in some embodiments then used to make a probabilistic inference about whether the monitored individual is suffering from endometriosis and whether the monitored individual has in the past been diagnosed with endometriosis. The former prediction may be described as an inferred endometriosis prediction and the latter as an inferred endometriosis diagnosis history prediction. The noted predictions, while being probabilistic in nature and thus not sufficient for reliable endometriosis-based action recommendation, nevertheless provide powerful tools for determining whether to generate a predicted endometriosis-based recommendation for the monitored individual and, if a determination is made that a predicted endometriosis-based recommendation should be generated for the monitored individual, to generate the noted predicted endometriosis-based recommendation for the monitored individual.

An exemplary application of various embodiments of the present invention relates to providing a method that analyzes member symptoms and provides a member with an early indication of whether the member has endometriosis, as well as whether the member should see a specialist who can investigate to confirm/rule out endometriosis. In some embodiments, the following operations are performed: particular initial features (e.g., gender of the patient, most recent CA125 level of the patient, whether the patient has a family history of endometriosis, whether the patient has an ovarian cyst, whether a magnetic resonance imaging (MRI) scan has been performed on the patient, whether the patient has been prescribed birth control pills, whether the patient has taken female hormone medicine, whether the patient has a prior history of miscarriage, age of the patient, body mass index (BMI) of the patient, and whether the patient has a history of previous abdomen surgery) are selected for a patient; the initial features are processed using a first classification model to determine whether the patient is at risk of endometriosis or not based at least in part on whether different patterns (e.g., Pattern 1: Female, having symptoms, with no pills in drugs prescription, with no scans, which may lead to inferring not diagnosed yet; Pattern 2: Female, having symptoms with hormonal pills already prescribed, which may lead to inferring previous diagnosis; and/or the like) can be detected in patient data; and a set of endometriosis features are processed using a second classification model to select a recommendation category from a set of recommendation categories for the patient. The set of recommendation categories may in some embodiments include: a first gynae category characterized by age (teenager), no previous miscarriage, and past prescription of birth control or female hormones; a second egg freeze category characterized by age (above twenty), past diagnosis, and huge size of ovarian cyst; and third in vitro fertilization category characterized by previous miscarriage history.

### II. Definitions

The term “diagnostic feature value” may refer to a data construct that describes a feature value that can be used to map a corresponding monitored individual to a selected diagnostic profile classification. In some embodiments, each diagnostic feature value is associated with a diagnostic feature type. Examples of diagnostic feature values include a gender feature value for a gender feature type that describes a recorded gender of a monitored individual, a cancer antigen 125 (CA-125) level feature value having a CA-125 level feature type that describes a selected (e.g., a most recent) CA-125 measurement of a monitored individual, an endometriosis family history feature value having an endometriosis family history feature type that describes whether a monitored individual has a family history of endometriosis, an ovarian cyst feature value having an ovarian cyst feature type that describes whether a monitored individual is recorded to have an ovarian cyst and/or a large ovarian cyst, a medical scan feature value having a medical scan feature type that describes whether a monitored individual is recorded to have been subject to a magnetic resonance imaging (MRI) scan and/or to a UTS, a birth control prescription history feature value having a birth control prescription history feature type that describes whether a monitored individual is recorded to have been prescribed a birth control medication (e.g., within a defined time period), a latent symptom feature value having a latent symptom feature type that describes whether a monitored individual is recorded to have experienced a previous miscarriage, an age feature value having an age feature type that describes the recorded age of a monitored individual, a body mass index (BMI) feature value having a BMI feature type that describes a recorded BMI and/or a recorded BMI average (e.g., within a defined time period) of a monitored individual, and a previous abdomen surgery feature value having a previous abdomen surgery feature type that describes whether a monitored individual is recorded to have been subject to previous abdomen surgery (e.g., within a defined time period).

The term “defined diagnostic profile classification” may refer to a data construct that describes a group of diagnosis feature values for a group of diagnosis feature types, where association of a monitored individual with the defined diagnostic profile classification describes that the monitored individual is associated with the group of diagnosis feature values. In some embodiments, each defined diagnostic profile classification is associated with a group of diagnosis profile feature values that comprise a first subset of diagnosis profile feature values that are associated with at least a subset of the plurality of diagnostic feature types described above and a second subset of profile diagnosis feature values that are not associated with the plurality of diagnostic feature types described above. For example, given a set of n diagnostic feature values that are associated with n diagnostic profile feature types, a particular defined diagnostic profile classification may be associated with m diagnosis profile feature values for m diagnosis feature types, where m-a of the diagnosis profile feature types correspond to n-a of the diagnostic feature types, and a of the diagnosis profile feature types are independent of the n diagnostic feature types. In some embodiments, each defined diagnostic profile classification is associated with: (i) a demographic profile that describes, for each demographic feature type of at least one demographic feature type of the one or more demographic feature types, a corresponding demographic feature type value, (ii) a symptom history profile that describes, for each symptom history feature type of at least one symptom history feature type of the one or more symptom history feature types, a corresponding symptom history feature type value, and (iii) a treatment history profile that describes, for each treatment history feature type of at least one treatment history feature type of the one or more treatment history feature types, a corresponding treatment history feature type value.

The term “diagnosis classification machine learning model” may refer to a data construct that describes parameters, hyper-parameters, and/or defined operations of a model (e.g., a trained machine learning model, a rule-based machine learning model, a hybrid machine learning model, and/or the like) that is configured to process a plurality of diagnostic feature values (e.g., an n-dimensional vector comprising n diagnostic feature values) for a monitored individual to generate a model output value that can be used to select a defined diagnostic profile classification for the monitored individual. In some embodiments, the diagnostic classification machine learning model is a powerful supervised machine learning tool that can be trained without labeled training data describing desired target information yet can be configured to generate predictive outputs that can be used to probabilistically infer the desired target information. For example, the diagnostic classification machine learning model can generate predictions about whether a monitored individual is associated with an endometriosis diagnosis history (i.e., whether the monitored has in the past been diagnosed with endometriosis) and/or whether a monitored individual is suffering from endometriosis without being trained on labeled training data describing endometriosis diagnosis history labels and/or endometriosis diagnosis labels. To do so, the diagnostic classification machine learning model can be trained to map monitored individuals to defined diagnostic profile classifications instead of to target diagnosis history predictions and/or to target diagnosis predictions, and then the diagnostic profile classifications may be used to infer distributions for target diagnosis history predictions and/or to target diagnosis predictions that can be used to generating a predicted endometriosis-based recommendation for a monitored individual. In some embodiments, inputs to a diagnosis classification machine learning model include an n-dimensional vector comprising n diagnostic feature values, while outputs of a diagnosis classification machine learning model include a vector that describes, for each of a set of defined diagnosis profile classifications, the likelihood that the defined diagnosis profile classification is the selected diagnosis profile classification for an input monitored individual. In some embodiments, inputs to a diagnosis classification machine learning model include an n-dimensional vector comprising n diagnostic feature values, while outputs of a diagnosis classification machine learning model include a vector and/or an atomic value that describes the selected diagnosis profile classification for an input monitored individual.

The term “recommendation feature value” may refer to a data construct that describes a feature value of a monitored individual that is used as an input to a recommendation classification machine learning model to generate a selected recommendation profile for the monitored individual. In some embodiments, recommendation feature values for a monitored individual include at least a subset of the diagnostic feature values for the monitored individual, any diagnosis feature values of the selected diagnostic profile classification of the monitored individual that are independent of (e.g., that are not among) the diagnostic feature values for the monitored individual, and/or one or more exogenous recommendation feature values that are deemed important to generating an endometriosis-based action recommendation for the monitored individual. In some embodiments, the recommendation feature values for a monitored individual include at least one of an age feature value, a latent symptom feature value, a pill prescription feature value that describes whether the monitored individual has been prescribed birth control or female hormones, a previous abdomen surgery feature, an ovarian cyst feature size, the inferred endometriosis prediction, and the endometriosis diagnosis history prediction. In some embodiments, the recommendation feature values for a monitored individual may include the inferred endometriosis diagnosis history prediction for the selected diagnosis profile classification that is associated with the monitored individual. The inferred endometriosis diagnosis history prediction for a monitored individual may describe a Boolean value that describes whether the monitored individual is more likely than not to have in the past been diagnosed with endometriosis in the past. This value, while being probabilistic in nature and thus not sufficient for reliable determination about whether a monitored individual has been diagnosed with endometriosis, provides partial predictive insights to a recommendation classification machine learning in order to generate a predicted endometriosis-based recommendation for a monitored individual. In some embodiments, the inferred endometriosis diagnosis history prediction of a given defined diagnostic profile classification is determined based at least in part on the treatment history profile of a given defined diagnostic profile classification, where the treatment history profile of the given defined diagnostic profile classification may describe, for each treatment history feature type of at least one treatment history feature type of the one or more treatment history feature types, a corresponding treatment history feature type value. For example, the inferred endometriosis diagnosis history prediction of a given defined diagnostic profile classification may be associated with an affirmative endometriosis diagnosis history prediction if at least j of the treatment feature values described by the treatment history profile for the given defined diagnostic profile classification describe occurrence of endometriosis-based treatments. In some embodiments, given a sequence of t treatments associated with a monitored individual, the sequence is given to a inferred endometriosis diagnosis history prediction recurrent neural network machine learning model via t timesteps, where each of the t timesteps is configured to generate a hidden state that may be provided to a subsequent timestep, and where the inferred endometriosis diagnosis history prediction is determined based at least in part on a generated hidden state of a final timestep of the t timesteps of the inferred endometriosis diagnosis history prediction recurrent neural network machine learning model.

The term “defined recommendation profile classification” may refer to a data construct that describes a group of recommendation feature values for a group of recommendation feature types, where association of a monitored individual with the defined recommendation profile classification describes that the monitored individual is associated with the group of recommendation feature values. In some embodiments, each defined recommendation profile classification is associated with a group of recommendation profile feature values that comprise a first subset of recommendation profile feature values that are associated with at least a subset of the plurality of recommendation profile feature types described above and a second subset of recommendation profile feature values that are not associated with the plurality of recommendation profile feature types described above. For example, given a set of v recommendation feature values that are associated with v recommendation feature types, a particular defined recommendation profile classification may be associated with w recommendation profile feature values for w recommendation feature types, where w-x of the recommendation profile feature types correspond to w-x of the recommendation feature types, and x of the recommendation profile feature types are independent of the v recommendation profile feature types. An example of a defined recommendation profile classification is an early diagnosis recommendation profile is associated with an affirmative inferred endometriosis diagnosis history prediction and an early age demographic feature value (describing that monitored individuals associated with the noted defined recommendation profile classification have a defined early range of age, such as are in their teen ages). In some embodiments, the early diagnosis recommendation profile is associated with a second opinion action that describes that monitored individuals associated with the noted defined recommendation profile classification should get a second opinion from a qualified provider such as a gynecologist.

The term “recommendation classification machine learning model” may refer to a data construct that describes parameters, hyper-parameters, and/or defined operations of a model (e.g., a trained machine learning model, a rule-based machine learning model, a hybrid machine learning model, and/or the like) process a plurality of recommendation feature values (e.g., an v-dimensional vector comprising v recommendation feature values) for a monitored individual to generate a model output value that can be used to select a defined recommendation profile classification for the monitored individual. In some embodiments, the recommendation classification machine learning model is a powerful supervised machine learning tool that can be trained without labeled training data describing desired target information yet can be configured to generate predictive outputs that can be used to probabilistically infer the desired target information. For example, the recommendation classification machine learning model can generate predictions about recommended endometriosis-based actions for particular individuals without being trained on labeled training data describing ground-truth recommended endometriosis-based actions for the noted particular individuals. To do so, the recommendation classification machine learning model can be trained to map monitored individuals to defined recommendation profile classifications instead of to target recommendations, and then the recommendation profile classifications may be used to infer distributions for target recommendations. some embodiments, inputs to a diagnosis classification machine learning model include an v-dimensional vector comprising v recommendation feature values, while outputs of a recommendation classification machine learning model include a vector that describes, for each of a set of defined recommendation profile classifications, the likelihood that the defined recommendation profile classification is the selected recommendation profile classification for an input monitored individual. In some embodiments, inputs to a recommendation classification machine learning model include an v-dimensional vector comprising v recommendation feature values, while outputs of a recommendation classification machine learning model include a vector and/or an atomic value that describes the selected recommendation profile classification for an input monitored individual.

The term “inferred endometriosis diagnosis history prediction” may refer to a data construct that describes a describe a Boolean value that describes whether the monitored individual is more likely than not to have in the past been diagnosed with endometriosis in the past. This value, while being probabilistic in nature and thus not sufficient for reliable determination about whether a monitored individual has been diagnosed with endometriosis, provides partial predictive insights to a recommendation classification machine learning in order to generate a predicted endometriosis-based recommendation for a monitored individual. In some embodiments, the inferred endometriosis diagnosis history prediction of a given defined diagnostic profile classification is determined based at least in part on the treatment history profile of a given defined diagnostic profile classification, where the treatment history profile of the given defined diagnostic profile classification may describe, for each treatment history feature type of at least one treatment history feature type of the one or more treatment history feature types, a corresponding treatment history feature type value. For example, the inferred endometriosis diagnosis history prediction of a given defined diagnostic profile classification may be associated with an affirmative endometriosis diagnosis history prediction if at least j of the treatment feature values described by the treatment history profile for the given defined diagnostic profile classification describe occurrence of endometriosis-based treatments. In some embodiments, given a sequence of t treatments associated with a monitored individual, the sequence is given to a inferred endometriosis diagnosis history prediction recurrent neural network machine learning model via t timesteps, where each of the t timesteps is configured to generate a hidden state that may be provided to a subsequent timestep, and where the inferred endometriosis diagnosis history prediction is determined based at least in part on a generated hidden state of a final timestep of the t timesteps of the inferred endometriosis diagnosis history prediction recurrent neural network machine learning model.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a scripting language, a database query or search language, and/or a report writing language. In one or more example embodiments, a software component comprising instructions in one of the foregoing examples of programming languages may be executed directly by an operating system or other software components without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD), solid-state card (SSC), solid-state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read-only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises a combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

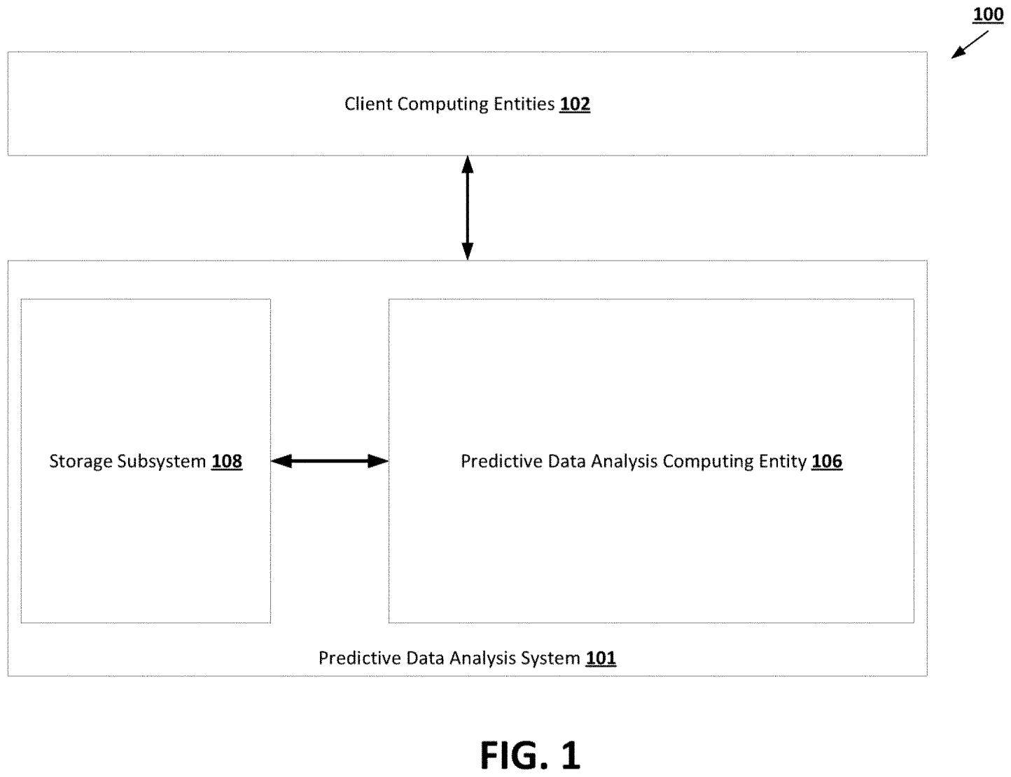

FIG. 1 is a schematic diagram of an example architecture 100 for performing predictive data analysis. The architecture 100 includes a predictive data analysis system 101 configured to receive predictive data analysis requests from client computing entities 102, process the predictive data analysis requests to generate predictions, provide the generated predictions to the client computing entities 102, and automatically perform prediction-based actions based at least in part on the generated predictions. An example of a prediction-based action that can be performed using the predictive data analysis system 101 is processing a request for determining a recommended endometriosis-based actions based at least in part on feature values (e.g., diagnostic feature values, recommendation feature values, and/or the like) for a monitored individual.

An exemplary application of various embodiments of the present invention relates to providing a method that analyzes member symptoms and provides a member with an early indication of whether the member has endometriosis, as well as whether the member should see a specialist who can investigate to confirm/rule out endometriosis. In some embodiments, the following operations are performed: particular initial features (e.g., gender of the patient, most recent CA125 level of the patient, whether the patient has a family history of endometriosis, whether the patient has an ovarian cyst, whether a magnetic resonance imaging (MRI) scan has been performed on the patient, whether the patient has been prescribed birth control pills, whether the patient has taken female hormone medicine, whether the patient has a prior history of miscarriage, age of the patient, body mass index (BMI) of the patient, and whether the patient has a history of previous abdomen surgery) are selected for a patient; the initial features are processed using a first classification model to determine whether the patient is at risk of endometriosis or not based at least in part on whether different patterns (e.g., Pattern 1: Female, having symptoms, with no pills in drugs prescription, with no scans, which may lead to inferring not diagnosed yet; Pattern 2: Female, having symptoms with hormonal pills already prescribed, which may lead to inferring previous diagnosis; and/or the like) can be detected in patient data; and a set of endometriosis features are processed using a second classification model to select a recommendation category from a set of recommendation categories for the patient. The set of recommendation categories may in some embodiments include: a first gynae category characterized by age (teenager), no previous miscarriage, and past prescription of birth control or female hormones; a second egg freeze category characterized by age (above twenty), past diagnosis, and huge size of ovarian cyst; and third in vitro fertilization category characterized by previous miscarriage history.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive predictive data analysis requests from one or more client computing entities 102, process the predictive data analysis requests to generate predictions corresponding to the predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform predictive data analysis as well as model definition data used by the predictive data analysis computing entity 106 to perform various predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

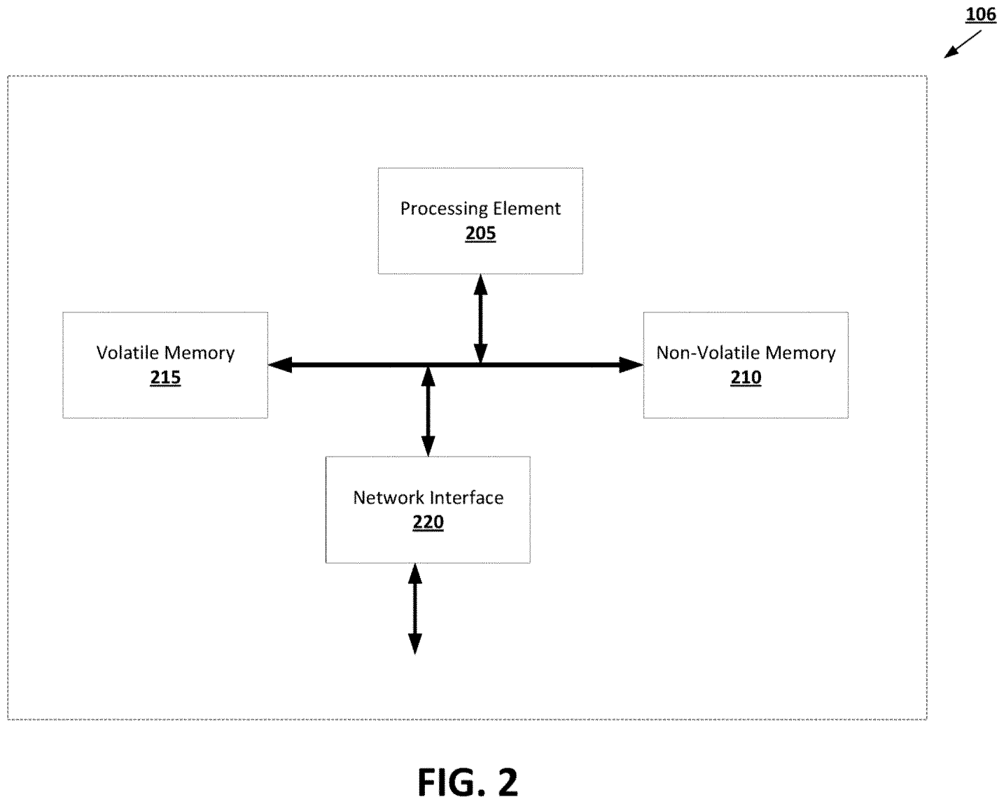

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application-specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### Exemplary Client Computing Entity

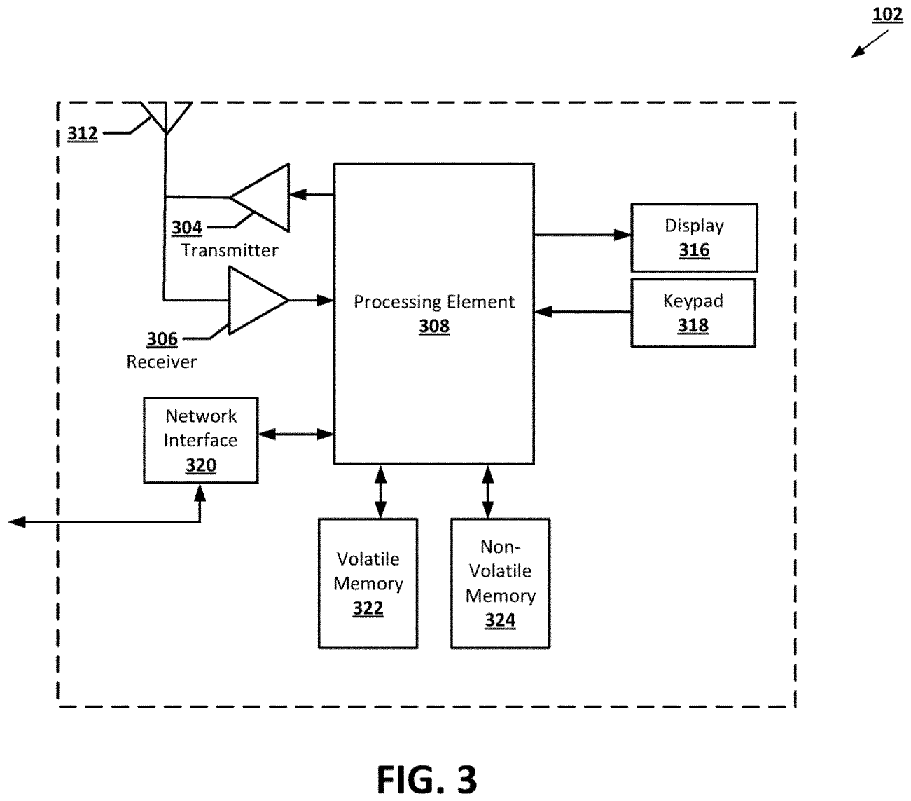

FIG. 3 provides an illustrative schematic representative of a client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As described below, various embodiments of the present invention introduce techniques for training diagnosis classification machine learning models and/or recommendation classification machine learning models without using target training data describing diagnosis labels and/or recommendation labels. By doing so, various embodiments of the present invention reduce the storage-wise complexity and thus improve storage-wise efficiency of predictive data analysis systems that are configured to train and use diagnosis classification machine learning models and/or recommendation classification machine learning models by reducing the amount of training data those systems need to store to train diagnosis classification machine learning models and/or recommendation classification machine learning models. Moreover, by enabling techniques for training diagnosis classification machine learning models and/or recommendation classification machine learning models without using target training data describing diagnosis labels and/or recommendation labels, various embodiments of the present invention enable accurate and efficient machine learning even in the absence of target training data describing diagnosis labels and/or recommendation labels, thus expanding the utility of the resulting models and the environments in which those models can be utilized. Accordingly, various embodiments of the present invention improve efficiency and reliability of predictive data analysis systems that are configured to train and use diagnosis classification machine learning models and/or recommendation classification machine learning models, and make important technical contributions to the field of predictive data analysis.

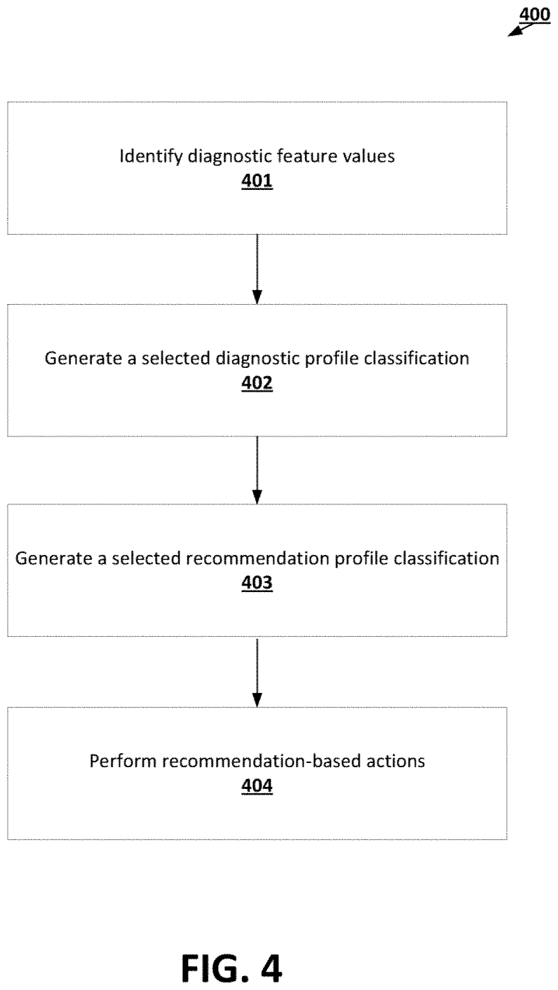

FIG. 4 is a flowchart diagram of an example process 400 for generating a predicted endometriosis-based recommendation for a monitored individual. Via the various steps/operations of the process 400, the predictive data analysis computing entity 106 is able to use diagnostic features generated based at least in part on medical history data for the monitored individual to generate recommendation features that can be used to generate the predicted endometriosis-based recommendation, thus performing transfer learning by mapping output of a diagnostic classification machine learning model to the output of a recommendation classification machine learning model.

The process 400 begins at step/operation 401 when the predictive data analysis computing entity 106 identifies one or more diagnostic feature values for the monitored individual. The monitored individual may be any individual with respect to whom one or more feature values are obtained. For example, the feature values for a monitored individual may be determined based at least in part on medical history data (e.g., medical claim history data) for the monitored individual, based at least in part on data obtained via continuous biometric monitoring of the monitored individual, based at least in part on demographic data associated with the monitored individual, and/or the like.

In some embodiments, a diagnostic feature value may refer to a feature value that can be used to map a corresponding monitored individual to a selected diagnostic profile classification. In some embodiments, each diagnostic feature value is associated with a diagnostic feature type. Examples of diagnostic feature values include a gender feature value for a gender feature type that describes a recorded gender of a monitored individual, a cancer antigen 125 (CA-125) level feature value having a CA-125 level feature type that describes a selected (e.g., a most recent) CA-125 measurement of a monitored individual, an endometriosis family history feature value having an endometriosis family history feature type that describes whether a monitored individual has a family history of endometriosis, an ovarian cyst feature value having an ovarian cyst feature type that describes whether a monitored individual is recorded to have an ovarian cyst and/or a large ovarian cyst, a medical scan feature value having a medical scan feature type that describes whether a monitored individual is recorded to have been subject to a magnetic resonance imaging (MRI) scan and/or to a UTS, a birth control prescription history feature value having a birth control prescription history feature type that describes whether a monitored individual is recorded to have been prescribed a birth control medication (e.g., within a defined time period), a latent symptom feature value having a latent symptom feature type that describes whether a monitored individual is recorded to have experienced a previous miscarriage, an age feature value having an age feature type that describes the recorded age of a monitored individual, a body mass index (BMI) feature value having a BMI feature type that describes a recorded BMI and/or a recorded BMI average (e.g., within a defined time period) of a monitored individual, and a previous abdomen surgery feature value having a previous abdomen surgery feature type that describes whether a monitored individual is recorded to have been subject to previous abdomen surgery (e.g., within a defined time period).

In some embodiments, diagnostic feature values of a monitored individual include: one or more demographic feature values, one or more symptom history feature types, one or more measurement history feature values, and/or one or more treatment history feature values. A demographic feature value may describe one or more feature values of a corresponding monitored individual that are independent of the medical history of the corresponding monitored individual. Examples of demographic feature values include a gender feature value, an age feature value, a location feature value, and/or the like. A symptom history feature values may describe one or more feature values of a corresponding monitored individual that describe whether the monitored individual is recorded to have experienced one or more symptoms/signs/correlating factors of an endometriosis condition. Examples of symptom history feature values include an endometriosis family history feature value, an ovarian cyst feature type, and/or the like. A measurement history feature value may describe one or more feature values of a corresponding monitored individual that describe one or more measurements associated with the corresponding monitored individual. Examples of measurement history feature values include a CA-125 level feature value, a BMI feature value, and/or the like. A treatment history feature value may describe one or more feature values of a corresponding monitored individual that describes one or more medical interventions performed with respect to the corresponding monitored individual. Examples of treatment history feature values include a medical scan feature value, a birth control prescription history feature type, a previous abdomen surgery feature type, and/or the like.

At step/operation 402, the predictive data analysis computing entity 106 is configured to process the plurality of diagnostic features to generate a selected diagnostic profile classification for the monitored individual. In some embodiments, the predictive data analysis computing entity 106 uses a diagnostic classification machine learning model to process the plurality of diagnostic features to generate the selected diagnostic profile classification. In some embodiments, the selected diagnostic profile classification is a selected one of a plurality of defined diagnostic profile classification.

A defined diagnostic profile classification may be characterized by a group of diagnosis feature values for a group of diagnosis feature types, where association of a monitored individual with the defined diagnostic profile classification describes that the monitored individual is associated with the group of diagnosis feature values. In some embodiments, each defined diagnostic profile classification is associated with a group of diagnosis profile feature values that comprise a first subset of diagnosis profile feature values that are associated with at least a subset of the plurality of diagnostic feature types described above and a second subset of profile diagnosis feature values that are not associated with the plurality of diagnostic feature types described above. For example, given a set of n diagnostic feature values that are associated with n diagnostic profile feature types, a particular defined diagnostic profile classification may be associated with m diagnosis profile feature values for m diagnosis feature types, where m-a of the diagnosis profile feature types correspond to n-a of the diagnostic feature types, and a of the diagnosis profile feature types are independent of the n diagnostic feature types.

In some embodiments, each defined diagnostic profile classification is associated with: (i) a demographic profile that describes, for each demographic feature type of at least one demographic feature type of the one or more demographic feature types, a corresponding demographic feature type value, (ii) a symptom history profile that describes, for each symptom history feature type of at least one symptom history feature type of the one or more symptom history feature types, a corresponding symptom history feature type value, and (iii) a treatment history profile that describes, for each treatment history feature type of at least one treatment history feature type of the one or more treatment history feature types, a corresponding treatment history feature type value.

An example of a defined diagnostic profile classification is a defined diagnostic profile classification that is associated with the following diagnosis feature values: (i) a female gender feature value, (ii) at least one affirmative symptom history feature value for a symptom history feature type that describes presence of an endometriosis symptom, (iii) a negative pregnancy pill feature value that describes absence of pregnancy pill prescription in the medical history of monitored individuals associated with the defined diagnostic profile classification, and (iv) a negative endometriosis diagnosis prediction that describes that monitored individuals associated with the defined diagnostic profile classification have not been diagnosed with endometriosis. In the noted example, the noted defined diagnostic profile classification has at least one diagnosis feature value (i.e., the negative endometriosis diagnosis prediction) that is not a feature value that is not one of the exemplary diagnostic feature values discussed. Thus, by mapping a monitored individual to the defined diagnostic profile classification, a computing entity can use diagnostic feature values of a particular monitored individual to generate a prediction about the negative endometriosis diagnosis prediction of the particular monitored individual.

Another example of a defined diagnostic profile classification is a defined diagnostic profile classification that is associated with the following diagnosis feature values: (i) a female gender feature value, (ii) at least one affirmative symptom history feature value for a symptom history feature type that describes presence of an endometriosis symptom, (iii) an affirmative pregnancy pill feature value that describes absence of pregnancy pill prescriptions in the monitored individuals associated with the defined diagnostic profile classification, and (iv) an affirmative endometriosis diagnosis prediction that describes that monitored individuals associated with the defined diagnostic profile classification have been diagnosed with endometriosis. In the noted example, the noted defined diagnostic profile classification has at least one diagnosis feature value (i.e., the affirmative endometriosis diagnosis prediction) that is not a feature value that is not one of the exemplary diagnostic feature values discussed. Thus, by mapping a monitored individual to the defined diagnostic profile classification, a computing entity can use diagnostic feature values of a particular monitored individual to generate a prediction about the affirmative endometriosis diagnosis prediction of the particular monitored individual.

In some embodiments, to map a monitored individual having n diagnostic feature values to a selected one of p defined diagnostic profile classifications, the predictive data analysis computing entity 106 may perform the following operations: (i) process the n diagnostic feature values using a diagnostic classification machine learning model to generate, for each defined diagnostic profile classification of the p defined diagnostic profile classifications, a classification score that describes the predicted likelihood that the monitored individual is associated with the defined diagnostic profile classification, (ii) identify a selected subset of the p defined diagnostic profile classifications, where each defined diagnostic profile classification in the selected subset is associated with a group of diagnosis feature values that do not contradict the n diagnostic feature values of the monitored individual, and (iii) map the monitored individual to a defined diagnostic profile classification of the p defined diagnostic profile classifications that has the highest classification scores of all of the defined diagnostic profile classifications that are in the selected subset.

In some embodiments, to map a monitored individual having n diagnostic feature values to a selected one of p defined diagnostic profile classifications, the predictive data analysis computing entity 106 may perform the following operations: (i) identify c diagnostic profile classification combinations, where each diagnostic profile classification combination comprises a defined combination of one or more of the p defined diagnostic profile classifications, (ii) for each of the diagnostic profile classification combination of the c diagnostic profile classification combinations, identify a diagnosis classification machine learning model of c diagnosis classification machine learning models, (iii) identify a selected subset of the p defined diagnostic profile classifications, where each defined diagnostic profile classification in the selected subset is associated with a group of diagnosis feature values that do not contradict the n diagnostic feature values of the monitored individual, (iv) determine a selected diagnostic profile classification combination of the c diagnostic profile classification combinations that comprises (e.g., consists of, consists essentially of, includes, and/or the like) the selected subset, (v) process the n diagnostic feature values using a selected diagnosis classification machine learning model of c diagnosis classification machine learning models that is associated with the selected diagnostic profile classification combination to determine, for each defined diagnostic profile classification of the defined diagnostic profile classifications in the selected diagnostic profile classification combination, a classification score that describes the predicted likelihood that the monitored individual is associated with the defined diagnostic profile classification, and (vi) map the monitored individual to the defined diagnostic profile classification that is associated with a highest classification score.

In some embodiments, to map a monitored individual having n diagnostic feature values to a selected one of p defined diagnostic profile classifications, the predictive data analysis computing entity 106 may perform the following operations: (i) identify p diagnostic classification machine learning models, where each diagnostic classification machine learning model is associated with a defined diagnostic profile classification of the p defined diagnostic profile classifications, (ii) identify a selected subset of the p defined diagnostic profile classifications, where each defined diagnostic profile classification in the selected subset is associated with a group of diagnosis feature values that do not contradict the n diagnostic feature values of the monitored individual, (iii) for each defined diagnostic profile classification in the selected subset, process the n diagnostic feature values using the diagnostic classification machine learning model for the defined diagnostic profile classification to generate a classification score that describes a classification score that describes a predicted likelihood that the monitored individual is associated with the defined diagnostic profile classification, and (vi) map the monitored individual to a defined diagnostic profile classification of the p defined diagnostic profile classifications that has the highest classification scores of all of the defined diagnostic profile classifications that are in the selected subset.

In some embodiments, all of the three techniques noted in the preceding three paragraphs are combined using an ensemble learning approach to generate a final selected diagnostic profile classification for the monitored individual. For example, in some embodiments, each candidate defined diagnostic profile classification of a technique that is the defined diagnostic profile classification to which the monitored individual is mapped after the final step/operation of the technique is assigned a weight value (e.g., a trained weight value) based at least in part on a credibility score of the technique, and then the selected diagnostic profile classification is selected as the defined diagnostic profile classification having the highest weight value. In some embodiments, the weight values are generated during training and/or during a testing/validation of the ensemble learning framework.

In some embodiments, the diagnostic classification machine learning model may be configured to process a plurality of diagnostic feature values (e.g., an n-dimensional vector comprising n diagnostic feature values) for a monitored individual to generate a model output value that can be used to select a defined diagnostic profile classification for the monitored individual. In some embodiments, the diagnostic classification machine learning model is a powerful supervised machine learning tool that can be trained without labeled training data describing desired target information yet can be configured to generate predictive outputs that can be used to probabilistically infer the desired target information. For example, the diagnostic classification machine learning model can generate predictions about whether a monitored individual is associated with an endometriosis diagnosis history (i.e., whether the monitored has in the past been diagnosed with endometriosis) and/or whether a monitored individual is suffering from endometriosis without being trained on labeled training data describing endometriosis diagnosis history labels and/or endometriosis diagnosis labels. To do so, the diagnostic classification machine learning model can be trained to map monitored individuals to defined diagnostic profile classifications instead of to target diagnosis history predictions and/or to target diagnosis predictions, and then the diagnostic profile classifications may be used to infer distributions for target diagnosis history predictions and/or to target diagnosis predictions that can be used to generating a predicted endometriosis-based recommendation for a monitored individual.

To illustrate an exemplary application of the above-described point, consider a predictive task that seeks to recommend endometriosis-based actions (e.g., endometriosis-based medical interventions) for those monitored individuals that are likely suffering from endometriosis without labeled data describing whether particular monitored individuals are suffering from endometriosis and/or without labeled data describing what recommended endometriosis-based actions are for particular monitored individuals that are suffering from endometriosis. To do so, the diagnostic classification machine learning model may be trained to map a monitored individual to a selected diagnostic profile classification which can be based at least in part on medical history data for the monitored individual (and thus may be readily determinable during training data preparation process), where the selected diagnostic profile classification for the monitored individual is in some embodiments then used to make a probabilistic inference about whether the monitored individual is suffering from endometriosis and whether the monitored individual has in the past been diagnosed with endometriosis. The former prediction may be described as an inferred endometriosis prediction and the latter as an inferred endometriosis diagnosis history prediction. The noted predictions, while being probabilistic in nature and thus not sufficient for reliable endometriosis-based action recommendation, nevertheless provide powerful tools for determining whether to generate a predicted endometriosis-based recommendation for the monitored individual and, if a determination is made that a predicted endometriosis-based recommendation should be generated for the monitored individual, to generate the noted predicted endometriosis-based recommendation for the monitored individual. Using the inferred endometriosis predictions and the inferred endometriosis diagnosis history predictions to generate predicted endometriosis-based recommendations is described in greater detail below with reference to step/operation 403.

By using step/operation 402, as described below, various embodiments of the present invention introduce techniques for training diagnosis classification machine learning models and/or recommendation classification machine learning models without using target training data describing diagnosis labels and/or recommendation labels. By doing so, various embodiments of the present invention reduce the storage-wise complexity and thus improve storage-wise efficiency of predictive data analysis systems that are configured to train and use diagnosis classification machine learning models and/or recommendation classification machine learning models by reducing the amount of training data those systems need to store to train diagnosis classification machine learning models and/or recommendation classification machine learning models. Moreover, by enabling techniques for training diagnosis classification machine learning models and/or recommendation classification machine learning models without using target training data describing diagnosis labels and/or recommendation labels, various embodiments of the present invention enable accurate and efficient machine learning even in the absence of target training data describing diagnosis labels and/or recommendation labels, thus expanding the utility of the resulting models and the environments in which those models can be utilized. Accordingly, various embodiments of the present invention improve efficiency and reliability of predictive data analysis systems that are configured to train and use diagnosis classification machine learning models and/or recommendation classification machine learning models, and make important technical contributions to the field of predictive data analysis.

At step/operation 403, the predictive data analysis computing entity 106 generates the predicted endometriosis-based recommendation for the monitored individual based at least in part on the selected diagnostic profile classification for the monitored individual. In some embodiments, generating the predicted endometriosis-based recommendation for the monitored individual comprises, in response to determining that the inferred endometriosis prediction for the selected diagnostic profile classification is an affirmative inferred endometriosis prediction: (i) determining a plurality of recommendation feature values for the monitored individual based at least in part on the plurality of diagnostic feature values for the monitored individual and the inferred endometriosis diagnosis history prediction for the selected diagnostic profile classification, (ii) determining, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action, and (iii) determining the predicted endometriosis-based recommendation based at least in part on the recommended endometriosis-based action for the selected recommendation profile classification.

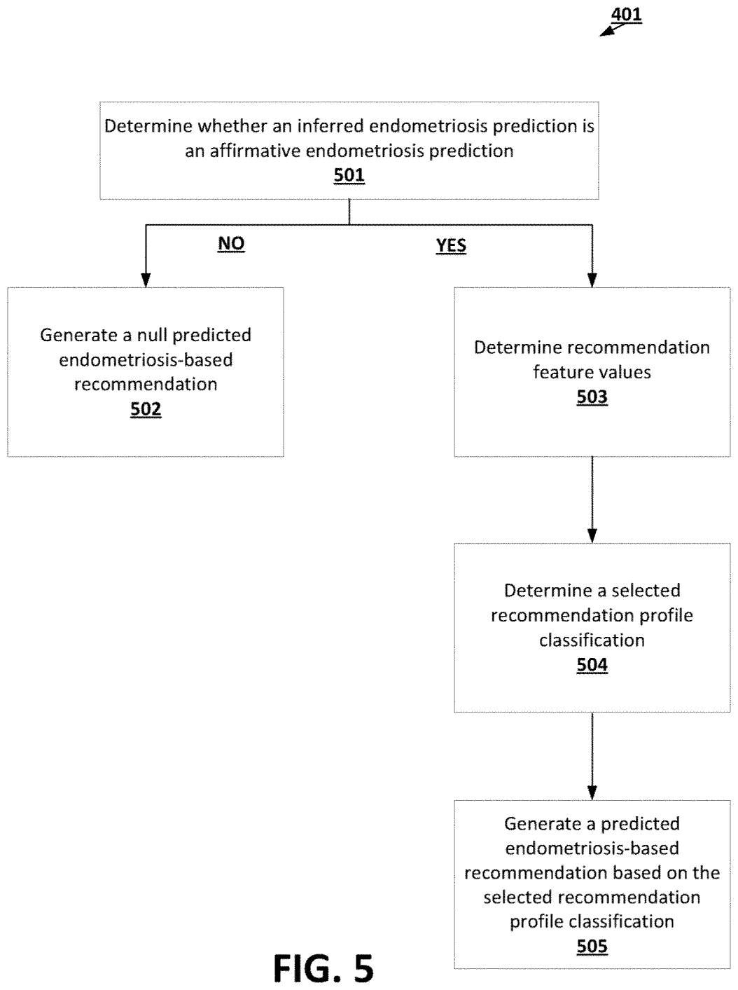

In some embodiments, step/operation 403 may be performed in accordance with the process that is depicted in FIG. 5. The process that is depicted in FIG. 5 begins at step/operation 501 when the predictive data analysis computing entity 106 determines whether the inferred endometriosis prediction for the selected diagnostic profile classification is an affirmative inferred endometriosis prediction. At step/operation 502, in response to determining that the inferred endometriosis prediction for the selected diagnostic profile classification is not an affirmative inferred endometriosis prediction, the predictive data analysis computing entity 106 generates a null predicted endometriosis-based recommendation that does not describe any recommended endometriosis-based actions.

As described above, in some embodiments, an inferred endometriosis prediction describes a Boolean value determined based at least in part on a selected diagnostic classification for a monitored individual that describes whether the monitored individual is more likely than not to suffer from endometriosis. This value, while being probabilistic in nature and thus not sufficient for reliable determination about whether a monitored individual is suffering from endometriosis, is used to make a partial predictive inference that is used to filter out monitored individuals for whom no recommended endometriosis-based actions should be generated. Accordingly, in some embodiments, if the inferred endometriosis prediction for the selected diagnostic profile classification is a negative inferred endometriosis prediction that describes that a corresponding monitored individual is likely to not suffer from endometriosis, then a null predicted endometriosis-based recommendation that does not describe any recommended endometriosis-based actions is generated for the noted corresponding monitored individual. However, if the inferred endometriosis prediction for the selected diagnostic profile classification is an affirmative inferred endometriosis prediction that describes that a corresponding monitored individual is likely to suffer from endometriosis, then a non-null predicted endometriosis-based recommendation that describes at least one recommended endometriosis-based action is generated for the noted corresponding monitored individual.

In response to determining that the inferred endometriosis prediction for the selected diagnostic profile classification is an affirmative inferred endometriosis prediction, the predictive data analysis computing entity 106 performs steps/operations 503-505. At step/operation 503, the predictive data analysis computing entity 106 determines a plurality of recommendation feature values for the recommendation feature values for the monitored individual based at least in part on the plurality of diagnostic feature values for the monitored individual and the inferred endometriosis diagnosis history prediction for the selected diagnostic profile classification.

In some embodiments, a recommendation feature value is a feature value of a monitored individual that is used as an input to a recommendation classification machine learning model to generate a selected recommendation profile for the monitored individual. In some embodiments, recommendation feature values for a monitored individual include at least a subset of the diagnostic feature values for the monitored individual, any diagnosis feature values of the selected diagnostic profile classification of the monitored individual that are independent of (e.g., that are not among) the diagnostic feature values for the monitored individual, and/or one or more exogenous recommendation feature values that are deemed important to generating an endometriosis-based action recommendation for the monitored individual. In some embodiments, the recommendation feature values for a monitored individual include at least one of an age feature value, a latent symptom feature value, a pill prescription feature value that describes whether the monitored individual has been prescribed birth control or female hormones, a previous abdomen surgery feature, an ovarian cyst feature size, the inferred endometriosis prediction, and the endometriosis diagnosis history prediction.

As described above, the recommendation feature values for a monitored individual may include the inferred endometriosis diagnosis history prediction for the selected diagnosis profile classification that is associated with the monitored individual. The inferred endometriosis diagnosis history prediction for a monitored individual may describe a Boolean value that describes whether the monitored individual is more likely than not to have in the past been diagnosed with endometriosis in the past. This value, while being probabilistic in nature and thus not sufficient for reliable determination about whether a monitored individual has been diagnosed with endometriosis, provides partial predictive insights to a recommendation classification machine learning in order to generate a predicted endometriosis-based recommendation for a monitored individual. In some embodiments, the inferred endometriosis diagnosis history prediction of a given defined diagnostic profile classification is determined based at least in part on the treatment history profile of a given defined diagnostic profile classification, where the treatment history profile of the given defined diagnostic profile classification may describe, for each treatment history feature type of at least one treatment history feature type of the one or more treatment history feature types, a corresponding treatment history feature type value. For example, the inferred endometriosis diagnosis history prediction of a given defined diagnostic profile classification may be associated with an affirmative endometriosis diagnosis history prediction if at least j of the treatment feature values described by the treatment history profile for the given defined diagnostic profile classification describe occurrence of endometriosis-based treatments. In some embodiments, given a sequence of t treatments associated with a monitored individual, the sequence is given to a inferred endometriosis diagnosis history prediction recurrent neural network machine learning model via t timesteps, where each of the t timesteps is configured to generate a hidden state that may be provided to a subsequent timestep, and where the inferred endometriosis diagnosis history prediction is determined based at least in part on a generated hidden state of a final timestep of the t timesteps of the inferred endometriosis diagnosis history prediction recurrent neural network machine learning model.

At step/operation 504, the predictive data analysis computing entity 106 processes the plurality of recommendation feature values for the monitored individual using the recommendation classification machine learning model to generate a selected recommendation profile classification of r recommendation profile classifications for the monitored individual. Exemplary embodiments of defined recommendation profile classifications and recommendation classification machine learning models are described below.

A defined recommendation profile classification may be characterized by a group of recommendation feature values for a group of recommendation feature types, where association of a monitored individual with the defined recommendation profile classification describes that the monitored individual is associated with the group of recommendation feature values. In some embodiments, each defined recommendation profile classification is associated with a group of recommendation profile feature values that comprise a first subset of recommendation profile feature values that are associated with at least a subset of the plurality of recommendation profile feature types described above and a second subset of recommendation profile feature values that are not associated with the plurality of recommendation profile feature types described above. For example, given a set of v recommendation feature values that are associated with v recommendation feature types, a particular defined recommendation profile classification may be associated with w recommendation profile feature values for w recommendation feature types, where w-x of the recommendation profile feature types correspond to w-x of the recommendation feature types, and x of the recommendation profile feature types are independent of the v recommendation profile feature types.

An example of a defined recommendation profile classification is an early diagnosis recommendation profile is associated with an affirmative inferred endometriosis diagnosis history prediction and an early age demographic feature value (describing that monitored individuals associated with the noted defined recommendation profile classification have a defined early range of age, such as are in their teen ages). In some embodiments, the early diagnosis recommendation profile is associated with a second opinion action that describes that monitored individuals associated with the noted defined recommendation profile classification should get a second opinion from a qualified provider such as a gynecologist.

Another example of a defined recommendation profile classification is a mid-age diagnosis recommendation profile that may be associated with an affirmative inferred endometriosis diagnosis history prediction and a mid-range age demographic feature value (describing that monitored individuals associated with the noted defined recommendation profile classification have a defined medium range of age, such as have 20 years or above). In some embodiments, the mid-age diagnosis recommendation profile is associated with an egg freeze action that describes that monitored individuals associated with the noted defined recommendation profile classification should freeze their eggs.

A yet another example of a defined recommendation profile classification is a latened symptom recommendation profile that may be associated with an affirmative latened symptom history feature value describing that monitored individuals associated with the noted defined recommendation profile classification have been recorded to have experienced past miscarriages. In some embodiments, the latened symptom recommendation is associated with an in vitro fertilization (IVF) specialization action that describes that monitored individuals associated with the noted defined recommendation profile classification should see an IVF specialist. In some embodiments, the inferred endometriosis diagnosis history prediction for a latened symptom recommendation profile is determined based at least in part on a symptom history profile of the given defined diagnostic profile. In some of the noted embodiments, the symptom history profile for the noted defined diagnostic profile describes, for each symptom history feature type of at least one symptom history feature type of the one or more symptom history feature types, a corresponding symptom history feature type value.

In some embodiments, to map a monitored individual having v recommendation feature values to a selected one of z defined recommendation profile classifications, the predictive data analysis computing entity 106 may perform the following operations: (i) process the v recommendation feature values using a recommendation classification machine learning model to generate, for each defined recommendation profile classification of the z defined recommendation profile classifications, a classification score that describes the predicted likelihood that the monitored individual is associated with the defined recommendation profile classification, (ii) identify a selected subset of the z defined recommendation profile classifications, where each defined recommendation profile classification in the selected subset is associated with a group of diagnosis feature values that do not contradict the v recommendation feature values of the monitored individual, and (iii) map the monitored individual to a defined recommendation profile classification of the z defined recommendation profile classifications that has the highest classification scores of all of the defined recommendation profile classifications that are in the selected subset.

In some embodiments, to map a monitored individual having v recommendation feature values to a selected one of z defined recommendation profile classifications, the predictive data analysis computing entity 106 may perform the following operations: (i) identify b recommendation profile classification combinations, where each recommendation profile classification combination comprises a defined combination of one or more of the z defined recommendation profile classifications, (ii) for each of the recommendation profile classification combination of the b recommendation profile classification combinations, identify a diagnosis classification machine learning model of c diagnosis classification machine learning models, (iii) identify a selected subset of the z defined recommendation profile classifications, where each defined recommendation profile classification in the selected subset is associated with a group of diagnosis feature values that do not contradict the v recommendation feature values of the monitored individual, (iv) determine a selected recommendation profile classification combination of the b recommendation profile classification combinations that comprises (e.g., consists of, consists essentially of, includes, and/or the like) the selected subset, (v) process the v recommendation feature values using a selected diagnosis classification machine learning model of c diagnosis classification machine learning models that is associated with the selected recommendation profile classification combination to determine, for each defined recommendation profile classification of the defined recommendation profile classifications in the selected recommendation profile classification combination, a classification score that describes the predicted likelihood that the monitored individual is associated with the defined recommendation profile classification, and (vi) map the monitored individual to the defined recommendation profile classification that is associated with a highest classification score.

In some embodiments, to map a monitored individual having v recommendation feature values to a selected one of z defined recommendation profile classifications, the predictive data analysis computing entity 106 may perform the following operations: (i) identify p recommendation classification machine learning models, where each recommendation classification machine learning model is associated with a defined recommendation profile classification of the z defined recommendation profile classifications, (ii) identify a selected subset of the z defined recommendation profile classifications, where each defined recommendation profile classification in the selected subset is associated with a group of diagnosis feature values that do not contradict the v recommendation feature values of the monitored individual, (iii) for each defined recommendation profile classification in the selected subset, process the v recommendation feature values using the recommendation classification machine learning model for the defined recommendation profile classification to generate a classification score that describes a classification score that describes a predicted likelihood that the monitored individual is associated with the defined recommendation profile classification, and (vi) map the monitored individual to a defined recommendation profile classification of the z defined recommendation profile classifications that has the highest classification scores of all of the defined recommendation profile classifications that are in the selected subset.

In some embodiments, all of the three techniques noted in the preceding three paragraphs are combined using an ensemble learning approach to generate a final selected recommendation profile classification for the monitored individual. For example, in some embodiments, each candidate defined recommendation profile classification of a technique that is the defined recommendation profile classification to which the monitored individual is mapped after the final step/operation of the technique is assigned a weight value (e.g., a trained weight value) based at least in part on a credibility score of the technique, and then the selected recommendation profile classification is selected as the defined recommendation profile classification having the highest weight value. In some embodiments, the weight values are generated during training and/or during a testing/validation of the ensemble learning framework.

In some embodiments, the recommendation classification machine learning model may be configured to process a plurality of recommendation feature values (e.g., an v-dimensional vector comprising v recommendation feature values) for a monitored individual to generate a model output value that can be used to select a defined recommendation profile classification for the monitored individual. In some embodiments, the recommendation classification machine learning model is a powerful supervised machine learning tool that can be trained without labeled training data describing desired target information yet can be configured to generate predictive outputs that can be used to probabilistically infer the desired target information. For example, the recommendation classification machine learning model can generate predictions about recommended endometriosis-based actions for particular individuals without being trained on labeled training data describing ground-truth recommended endometriosis-based actions for the noted particular individuals. To do so, the recommendation classification machine learning model can be trained to map monitored individuals to defined recommendation profile classifications instead of to target recommendations, and then the recommendation profile classifications may be used to infer distributions for target recommendations.

At step/operation 505, the predictive data analysis computing entity 106 generates the predicted endometriosis-based recommendation based at least in part on the recommended endometriosis-based action for the selected recommendation profile classification. In some embodiments, the recommended endometriosis-based action for the selected recommendation profile is associated with a plurality of provider nodes within a coverage graph, and the predicted endometriosis-based recommendation is performed based at least in part on each provider node that falls within an individualized subgraph of the coverage graph that is associated with a member node that is associated with the monitored individual. In some embodiments, a second diagnosis action is associated with a plurality of gynecologist provider nodes, an egg freeze action is associated with a plurality of egg freeze centers, and an IVF specialist action is associated with a plurality of IVF specialists.

By using step/operation 403, as described below, various embodiments of the present invention introduce techniques for training diagnosis classification machine learning models and/or recommendation classification machine learning models without using target training data describing diagnosis labels and/or recommendation labels. By doing so, various embodiments of the present invention reduce the storage-wise complexity and thus improve storage-wise efficiency of predictive data analysis systems that are configured to train and use diagnosis classification machine learning models and/or recommendation classification machine learning models by reducing the amount of training data those systems need to store to train diagnosis classification machine learning models and/or recommendation classification machine learning models. Moreover, by enabling techniques for training diagnosis classification machine learning models and/or recommendation classification machine learning models without using target training data describing diagnosis labels and/or recommendation labels, various embodiments of the present invention enable accurate and efficient machine learning even in the absence of target training data describing diagnosis labels and/or recommendation labels, thus expanding the utility of the resulting models and the environments in which those models can be utilized. Accordingly, various embodiments of the present invention improve efficiency and reliability of predictive data analysis systems that are configured to train and use diagnosis classification machine learning models and/or recommendation classification machine learning models, and make important technical contributions to the field of predictive data analysis.

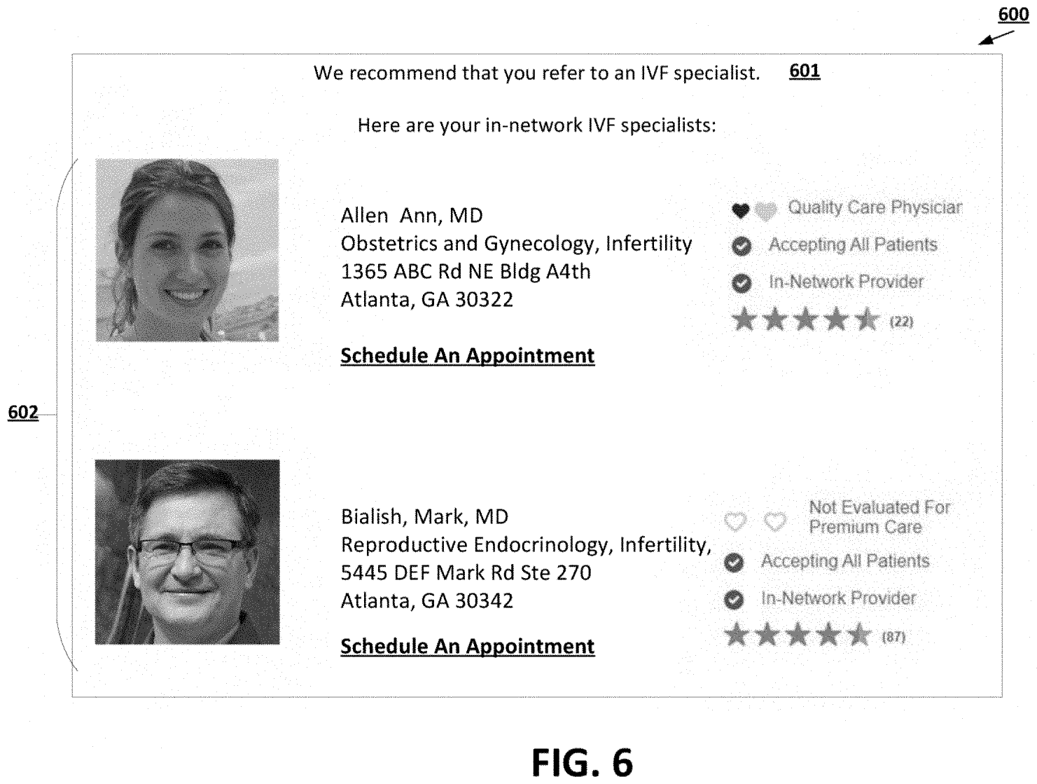

Returning to FIG. 4, at step/operation 404, the predictive data analysis computing entity 106 performs one or more recommendation-based actions based at least in part on the predicted endometriosis-based recommendation. In some embodiments, performing the one or more recommendation-based actions comprises generating user interface data for a recommendation user interface that describes the predicted endometriosis-based recommendation and/or a provider identifier associated with the predicted endometriosis-based recommendation, and enables scheduling an appointment with the provider identifier. An operational example of such a recommendation user interface 600 is depicted in FIG. 6. As depicted in FIG. 6, the recommendation user interface 600 describes the recommended endometriosis-based action 601 and the set of providers 602. As further depicted in FIG. 6, the recommendation user interface enables scheduling an appointment with a selected provider of the set of providers 602.

As described above, various embodiments of the present invention introduce techniques for training diagnosis classification machine learning models and/or recommendation classification machine learning models without using target training data describing diagnosis labels and/or recommendation labels. By doing so, various embodiments of the present invention reduce the storage-wise complexity and thus improve storage-wise efficiency of predictive data analysis systems that are configured to train and use diagnosis classification machine learning models and/or recommendation classification machine learning models by reducing the amount of training data those systems need to store to train diagnosis classification machine learning models and/or recommendation classification machine learning models. Moreover, by enabling techniques for training diagnosis classification machine learning models and/or recommendation classification machine learning models without using target training data describing diagnosis labels and/or recommendation labels, various embodiments of the present invention enable accurate and efficient machine learning even in the absence of target training data describing diagnosis labels and/or recommendation labels, thus expanding the utility of the resulting models and the environments in which those models can be utilized. Accordingly, various embodiments of the present invention improve efficiency and reliability of predictive data analysis systems that are configured to train and use diagnosis classification machine learning models and/or recommendation classification machine learning models, and make important technical contributions to the field of predictive data analysis.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for generating a predicted endometriosis-based recommendation for a monitored individual, the computer-implemented method comprising:
determining, using one or more processors, a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; and
in response to determining that the inferred endometriosis prediction for the selected diagnostic profile classification is an affirmative inferred endometriosis prediction, using the one or more processors:
determining a plurality of recommendation feature values for the monitored individual based at least in part on the plurality of diagnostic feature values for the monitored individual and the inferred endometriosis diagnosis history prediction for the selected diagnostic profile classification,
determining, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action,
determining the predicted endometriosis-based recommendation based at least in part on the recommended endometriosis-based action for the selected recommendation profile classification, and
performing one or more recommendation-based actions based at least in part on the predicted endometriosis-based recommendation.

2. The computer-implemented method of claim 1, wherein:
the plurality of diagnostic feature values are associated with a plurality of diagnostic feature types,
the plurality of diagnostic feature types comprise one or more demographic feature types, one or more symptom history feature types, and one or more treatment history feature types, and
each defined diagnostic profile classification is associated with: (i) a demographic profile that describes, for each demographic feature type of at least one demographic feature type of the one or more demographic feature types, a corresponding demographic feature type value, (ii) a symptom history profile that describes, for each symptom history feature type of at least one symptom history feature type of the one or more symptom history feature types, a corresponding symptom history feature type value, and (iii) a treatment history profile that describes, for each treatment history feature type of at least one treatment history feature type of the one or more treatment history feature types, a corresponding treatment history feature type value.

3. The computer-implemented method of claim 2, wherein the inferred endometriosis diagnosis history prediction of a given defined diagnostic profile classification is determined based at least in part on the treatment history profile of the given defined diagnostic profile classification.

4. The computer-implemented method of claim 1, wherein:
the plurality of defined recommendation profile classifications comprise an early diagnosis recommendation profile that is associated with a second opinion action, a mid-age diagnosis recommendation profile that is associated with an egg-freeze action, and a latened symptom recommendation profile that is associated with an in vitro fertilization specialist action.

5. The computer-implemented method of claim 4, wherein the early diagnosis recommendation profile is associated with an affirmative inferred endometriosis diagnosis history prediction and an early age demographic feature value.

6. The computer-implemented method of claim 4, wherein the mid-age diagnosis recommendation profile is associated with an affirmative inferred endometriosis diagnosis history prediction and a mid-range age demographic feature value.

7. The computer-implemented method of claim 4, wherein the latened symptom recommendation profile is associated with a latened symptom history feature value.

8. The computer-implemented method of claim 1, wherein the inferred endometriosis diagnosis history prediction for a given defined diagnostic profile classification is determined based at least in part on a symptom history profile of the given defined diagnostic profile classification.

9. The computer-implemented method of claim 8, wherein the symptom history profile for the given defined diagnostic profile describes, for each symptom history feature type of at least one symptom history feature type of one or more symptom history feature types, a corresponding symptom history feature type value.

10. The computer-implemented method of claim 1, wherein:
the recommended endometriosis-based action for the selected recommendation profile is associated with a plurality of provider nodes within a coverage graph, and
the predicted endometriosis-based recommendation is performed based at least in part on each provider node that falls within an individualized subgraph of the coverage graph that is associated with a member node that is associated with the monitored individual.

11. An apparatus for generating a predicted endometriosis-based recommendation for a monitored individual, the at least one memory and the program code configured to, with the at least one processor, cause the apparatus to at least:
determine, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; and
in response to determining that the inferred endometriosis prediction for the selected diagnostic profile classification is an affirmative inferred endometriosis prediction:
determine a plurality of recommendation feature values for the monitored individual based at least in part on the plurality of diagnostic feature values for the monitored individual and the inferred endometriosis diagnosis history prediction for the selected diagnostic profile classification,
determine, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action,
determine the predicted endometriosis-based recommendation based at least in part on the recommended endometriosis-based action for the selected recommendation profile classification, and
perform one or more recommendation-based actions based at least in part on the predicted endometriosis-based recommendation.

12. The apparatus of claim 11, wherein:
the plurality of diagnostic feature values are associated with a plurality of diagnostic feature types,
the plurality of diagnostic feature types comprise one or more demographic feature types, one or more symptom history feature types, and one or more treatment history feature types, and
each defined diagnostic profile classification is associated with: (i) a demographic profile that describes, for each demographic feature type of at least one demographic feature type of the one or more demographic feature types, a corresponding demographic feature type value, (ii) a symptom history profile that describes, for each symptom history feature type of at least one symptom history feature type of the one or more symptom history feature types, a corresponding symptom history feature type value, and (iii) a treatment history profile that describes, for each treatment history feature type of at least one treatment history feature type of the one or more treatment history feature types, a corresponding treatment history feature type value.

13. The apparatus of claim 12, wherein the inferred endometriosis diagnosis history prediction of a given defined diagnostic profile classification is determined based at least in part on the treatment history profile of the given defined diagnostic profile classification.

14. The apparatus of claim 11, wherein:
the plurality of defined recommendation profile classifications comprise an early diagnosis recommendation profile that is associated with a second opinion action, a mid-age diagnosis recommendation profile that is associated with an egg-freeze action, and a latened symptom recommendation profile that is associated with an in vitro fertilization specialist action.

15. The apparatus of claim 14, wherein the early diagnosis recommendation profile is associated with an affirmative inferred endometriosis diagnosis history prediction and an early age demographic feature value.

16. The apparatus of claim 14, wherein the mid-age diagnosis recommendation profile is associated with an affirmative inferred endometriosis diagnosis history prediction and a mid-range age demographic feature value.

17. The apparatus of claim 14, wherein the latened symptom recommendation profile is associated with a latened symptom history feature value.

18. The apparatus of claim 11, wherein the inferred endometriosis diagnosis history prediction for a given defined diagnostic profile classification is determined based at least in part on a symptom history profile of the given defined diagnostic profile classification.

19. The apparatus of claim 18, wherein the symptom history profile for the given defined diagnostic profile describes, for each symptom history feature type of at least one symptom history feature type of one or more symptom history feature types, a corresponding symptom history feature type value.

20. A computer program product for generating a predicted endometriosis-based recommendation for a monitored individual, the computer program product comprising at least one non-transitory computer readable storage medium having computer-readable program code portions stored therein, the computer-readable program code portions configured to:
determine, using a diagnostic classification machine learning model, and based at least in part on a plurality of diagnostic feature values for the monitored individual, a selected diagnostic profile classification of a plurality of defined diagnostic profile classifications, wherein each defined diagnostic profile classification is associated with an inferred endometriosis prediction and an inferred endometriosis diagnosis history prediction; and
in response to determining that the inferred endometriosis prediction for the selected diagnostic profile classification is an affirmative inferred endometriosis prediction:
determine a plurality of recommendation feature values for the monitored individual based at least in part on the plurality of diagnostic feature values for the monitored individual and the inferred endometriosis diagnosis history prediction for the selected diagnostic profile classification,
determine, using a recommendation classification machine learning model, and based at least in part on the plurality of recommendation feature values, a selected recommendation profile classification of a plurality of defined recommendation profile classifications, wherein each defined recommendation profile classification is associated with a recommended endometriosis-based action,
determine the predicted endometriosis-based recommendation based at least in part on the recommended endometriosis-based action for the selected recommendation profile classification, and
perform one or more recommendation-based actions based at least in part on the predicted endometriosis-based recommendation.
